SEQUENCE LISTING (2 sequences)

SEQ ID NO: 1 - DNA, 10 bp, Artificial Sequence
  Exemplary code unit sequence
aaaaattttt

SEQ ID NO: 2 - DNA, 10 bp, Artificial Sequence
  Exemplary code unit sequence
cccccggggg

(19) United States
(12) Patent Application Publication
(10) Pub. No.: US 2010/0092960 A1
(43) Pub. Date: Apr. 15, 2010

(54) Helicase-assisted sequencing with molecular beacons

(75) Inventor: Adrian Fehr, Los Altos, CA (US)

Correspondence Address: QUINE INTELLECTUAL PROPERTY LAW GROUP, P.C., P O BOX 458, ALAMEDA, CA, 94501, US

(73) Assignee: Pacific Biosciences of California, Inc., Menlo Park, CA (US)

(21) Appl. No.: 12/460,900

(22) Filed: Jul. 23, 2009

Related U.S. Application Data
(60) Provisional application No. 61/135,975, filed on Jul. 25, 2008.

(51) Int. Cl. C12Q 1/68

(52) U.S. Cl. 435/6

(57) ABSTRACT

Provided are compositions that include an at least partially single-stranded nucleic acid, at least one first molecular beacon, and an enzyme comprising a helicase activity, which enzyme is capable of removing the first molecular beacon from the single-stranded nucleic acid wherein the first molecular beacon is hybridized to a first complementary subsequence of the nucleic acid. Also provided are methods of determining the sequence of a template nucleic acid that include removing molecular beacons that are hybridized to the template from the template in a sequential manner using an enzyme that exhibits a helicase activity, detecting a sequence of fluorescent signals that is produced by the removal of a molecular beacons, and converting the sequence of fluorescent signals into nucleotide sequence information. Sequencing systems in which compositions and methods of the invention can be used are also provided.

### CROSS-REFERENCE TO RELATED APPLICATIONS

This application claims priority to and benefit of U.S. Provisional Patent Application 61/135,975, entitled, “Helicase-Assisted Sequencing With Molecular Beacons,” by Adrian Fehr, filed Jul. 25, 2008, the disclosure of which is incorporated herein in its entirety for all purposes.

### STATEMENT AS TO RIGHTS TO INVENTIONS MADE UNDER FEDERALLY SPONSORED RESEARCH AND DEVELOPMENT

This work was supported in part by grant number R01HG003710 from the National Human Genome Research Institute. The government has certain rights to this invention.

### FIELD OF THE INVENTION

This invention is in the field of nucleic acid sequencing. The invention relates to methods, compositions, and systems useful for determining the sequence of a nucleic acid.

### BACKGROUND OF THE INVENTION

Methods for determining the order of nucleotides in a nucleic acid have significantly accelerated biological research and discovery. Currently, nucleic acid sequence data are valuable in myriad applications in biological research and molecular medicine, including determining the hereditary factors in disease, in developing new methods to detect disease and to guide therapy (van de Vijver et al. (2002) “A gene-expression signature as a predictor of survival in breast cancer,” New England Journal of Medicine 347: 1999-2009), in drug development, and in providing a rational basis for personalized medicine. Obtaining and verifying sequence data for use in such analyses has made it necessary for sequencing technologies to undergo advancements to expand throughput, lower reagent and labor costs and to improve accuracy (See, e.g., Chan et al. (2005) “Advances in Sequencing Technology” (Review) Mutation Research 573: 13-40).

Nanopore sequencing is one method of determining the order of nucleotides on a single-stranded nucleic acid (Deamer et al. (2000) “Nanopores and nucleic acids: prospects for ultrarapid sequencing” Trends Biotechnol 18:147-51). The underlying principle of nanopore sequencing is that a single-stranded nucleic acid can be electrophoretically driven through a nano-scale pore, e.g., a pore of <2 nm in internal diameter, in such a way that the nucleic acid traverses the pore in a manner not unlike a thread passing through the eye of a needle. Because a translocating nucleic acid partially obstructs or blocks the nanopore, it alters the pore's electrical properties (Kasianowicz et al. (1996) “Characterization of individual polynucleotide molecules using a membrane channel” Proc Natl Acad Sci USA 93: 13770-13773). The translocation of a nucleic acid can then be detected and converted into an electrical signal, e.g., a change in current passing through the nanopore, which represents a direct reading of the nucleic acid sequence. Thus, unlike other high-throughput sequencing methods, e.g., single-molecule sequencing, pyrosequencing, sequencing-by-hybridization, etc., nanopore sequencing does not entail the amplification and/or chemical labeling of template nucleic acids.

Although the detection mode is extraordinarily sensitive and able to sense small differences in the base composition of the translocating nucleic acid, measurement of ionic conductivity alone is unlikely to achieve the resolution required for rapid sequential detection of each nucleotide in a DNA molecule. Furthermore, electrophoretic translocation can often move a nucleic acid through a nanopore too rapidly to permit the identification of individual bases.

To surmount these experimental difficulties, methods have been developed in which a “magnified” representation of, e.g., each nucleotide in the sequence of a nucleic acid template of interest, is produced (see, e.g., U.S. Pat. No. 6,723,513, entitled “Sequencing Method Using Magnifying Tags”, by Lexow), hybridized with fluorescently labeled probes, e.g., molecular beacons, and fed through a nanopore. Translocation of the single-stranded concatamer through the pore is slowed by the sequential “unzipping” of the molecular beacons from the concatamer, and the fluorescent signals generated by the removal of the molecular beacons from the concatamer can be detected with a high signal-to-noise ratio (Soni et al. (2007) “Progress toward Ultrafast DNA Sequencing Using Solid-State Nanopores” Clin Chem 53: 1996-2001). Advantageously, detection of the optical readout can be multiplexed to produce, e.g., high-density nanopore arrays that can increase the throughput of this sequencing method by several orders of magnitude.

However, the challenges associated with the fabrication and scalability of such high-density nanopore arrays retard the development of this system for high-throughput sequencing. What are needed in the art are methods for the efficient, sequential removal of labeled hybridization probes, e.g., molecular beacons, from a nucleic acid. What are also needed are compositions that can be beneficially used with the methods and sequencing systems that can integrate such methods and compositions for nucleic acid sequencing. In addition, such methods and systems are most beneficially automatable and/or capable of being multiplexed to permit high-throughput nucleic acid sequencing. The invention described herein fulfills these and other needs, as will be apparent upon review of the following.

### SUMMARY OF THE INVENTION

The present invention provides methods of sequencing a nucleic acid. In the methods, labeled hybridization probes are annealed to a nucleic acid of interest. Each probe is then removed, e.g., sequentially, from the nucleic acid by an enzyme or enzyme complex that exhibits probe-displacing activity, e.g., a helicase, a polymerase, or a ribosome. A sequence of transient signals is produced by the removal of the probes from the nucleic acid, and the signals are detected and converted into nucleotide sequence information, thus providing the sequence of the nucleic acid of interest. The sequencing methods provided herein can circumvent the need for costly, labor-intensive nucleic amplification and labeling, which can limit sequencing template sample production from matching the capacities of modern sequencing systems.

Thus, in a first aspect, the invention provides compositions that can be used in the methods. The compositions include a nucleic acid, e.g., an at least partially single-stranded nucleic acid, at least one labeled hybridization probe, and an enzyme that exhibits a helicase activity and/or a probe-displacing activity. The enzyme in the compositions can optionally be a DNA helicase, e.g., a uvrD, a Rep, a RecQ, a dnaB, a T4 gp41, or a T7 gp4, an RNA helicase, a DNA/RNA helicase, a DNA polymerase, an RNA polymerase, a reverse transcriptase, or a multi-enzyme complex, e.g., a ribosome. The enzyme of the compositions is capable of dissociating the labeled hybridization probe from the nucleic acid to produce a signal, e.g., a signal that can be converted into nucleotide sequence information. Optionally, the signal can be a fluorescent signal.

The hybridization probe of the compositions comprises a sequence complementary to a subsequence of the nucleic acid and can optionally be hybridized to the nucleic acid. Optionally, the probe can be a first molecular beacon that includes a first fluorophore that emits light at a first wavelength. In some embodiments, the compositions provided by the invention can optionally comprise at least one second labeled probe, e.g., at least one second molecular beacon that comprises a sequence complementary to a second subsequence of the nucleic acid and a second fluorophore at a first end that emits light at a second wavelength, e.g., a wavelength that is different from that emitted by the first fluorophore of the first molecular beacon. Optionally, the second molecular beacon can be hybridized to the second subsequence of the nucleic acid. Preferably, the first and second molecular beacons can be hybridized to the nucleic acid in a head-to-tail arrangement.

The nucleic acid of the compositions can optionally comprise an RNA or a DNA. The nucleic acid present in the composition can optionally comprise a sequence of interest, e.g., the sequence that is to be determined by methods provided herein, and the labeled probes that can anneal to the nucleic acid can each optionally comprise a short complementary subsequence of the sequence of interest. In other embodiments, the nucleic acid present in the composition comprises a coded representation of a sequence of interest, e.g., wherein each nucleotide in the sequence is optionally represented, e.g., in the nucleic acid of composition, by, e.g., one or more unique oligonucleotide. For example, a DNA included in the compositions can optionally comprise a concatenation of first code units and second code units. The first code units can comprise first unique oligonucleotide sequences and second code units can comprise second unique oligonucleotide sequences, such that each of four nucleotides in a sequence of a target nucleic acid is represented by a code unit pair. Thus, the sequence of code unit pairs of the concatenation represents the nucleotide sequence of the target nucleic acid.

The compositions of the invention can optionally be present on a planar surface, in a well, in a single-molecule reaction region, or in an observation volume. Optionally, the enzyme included in the compositions can be immobilized on the planar surface, in the well, in a single-molecule reaction region, or in an observation volume. Optionally, the compositions can include ATP, GTP, CTP, UTP, TTP, or a nucleotide analog.

In a related aspect, the invention provides methods of determining the sequence of a template nucleic acid, e.g., methods in which the above compositions can be used. The methods include hybridizing one or more labeled hybridization probes to a template nucleic acid and dissociating the probes from the template, e.g., in a sequential manner, with an enzyme that exhibits probe-displacing activity, e.g., a helicase, to produce a signal. The methods also include detecting the signal or sequence of signals, e.g., fluorescent signal or sequence of signals that is produced by the removal of the probes from the template, and converting the signal or sequence of signals, e.g., fluorescent signal or signals, into nucleotide sequence information, thus determining the sequence of the template nucleic acid.

Hybridizing labeled probes to a template nucleic acid can optionally include hybridizing molecular beacons to the template, e.g., in a head-to-tail arrangement. Providing the template can optionally include providing a single-stranded nucleic acid. Providing a single-stranded nucleic acid can optionally include converting a target nucleotide sequence into a concatenation of first code units and second code units, as described previously. Converting the target nucleic acid into a concatenation of first and second code units can optionally comprise any of the methods described herein. The oligonucleotide sequences of the first and second code units can optionally be about 10 nucleotides long.

The invention provides a second set of methods of determining the sequence of a template nucleic acid. These methods include providing a reaction mix comprising a thermostable enzyme that exhibits probe-displacing activity, e.g., Taq polymerase, and one or more labeled hybridization probes annealed to the template. The methods include dissociating the probes from the template with the enzyme to produce a signal, detecting the signal or a sequence of signals, and converting the signal or sequence of signals into nucleotide sequence information. The temperature of the reaction mix is then increased to dissociate the remaining probes from the template and to release the enzyme from the template and lowered again to allow rehybridization of the probes to the template.

Relatedly, the invention provides sequencing systems that include a reaction region, which contains a template nucleic acid to which a set of molecular beacons has been hybridized, e.g., in a head-to-tail fashion, and an enzyme that comprises a helicase and/or probe-displacing activity, e.g., an enzyme capable of sequential removal of the molecular beacons from the template nucleic acid. The systems also include a detector configured to detect a sequence of fluorescent signals produced by the sequential removal of the molecular beacons by the helicase in the reaction region and a conversion module that is capable of converting the sequence of fluorescent signals into nucleotide sequence information. Such systems can optionally include detectors, array readers, excitation light sources, one or more output devices, such as a printer and/or a monitor to display results, and the like.

Kits are also a feature of the invention. The present invention provides kits that incorporate the compositions of the invention, including one or more probe-displacing enzyme, e.g., a DNA helicase, e.g., uvrD, a Rep, a RecQ, a dnaB, a T4 gp41, or a T7 gp4, an RNA helicase, a DNA/RNA helicase, a DNA polymerase, an RNA polymerase, or a reverse transcriptase, that can be packaged in a fashion to enable its use. The kits of the invention optionally include additional useful reagents, such as a control template nucleic acids, buffer solutions and/or salt solutions, including, e.g., divalent metal ions, i.e., Mg⁺⁺, Mn⁺⁺ and/or Fe⁺⁺, molecular beacons, e.g., to prepare template nucleic acids for sequencing, etc. Such kits also typically include a container to hold the kit components, instructions for use of the compositions, e.g., to sequence a template nucleic acid.

Those of skill in the art will appreciate that the methods and compositions provided by the invention can be used alone or in combination. Systems that include modules for the production of DNA concatenations of code units and/or hybridization of molecular beacons to such DNA concatenations are also a feature of the invention and can be used in combination with the sequencing systems described above.

### BRIEF DESCRIPTION OF THE DRAWINGS

FIG. 1 provides a schematic depiction of a “head-to-tail arrangement” of molecular beacons hybridized to a single-stranded nucleic acid.

FIG. 2A shows how a nucleotide sequence of a template DNA can be represented as a string of binary values. FIG. 2B shows how the string of binary values can be represented as a DNA concatamer comprising first and second code units

FIG. 3 provides a schematic depiction of molecular beacons hybridized to the concatamer of FIG. 2.

FIG. 4 provides a schematic depiction of a method of determining the sequence of a nucleic acid template by using a helicase to sequentially “unzip” molecular beacons from the concatamer of FIG. 3.

FIG. 5 illustrates a method of determining the sequence of a nucleic acid via enzymatic removal of a labeled hybridization probe from a nucleic acid. The figure also depicts related compositions.

FIG. 6 illustrates a method of determining the sequence of a nucleic acid via the enzymatic removal of molecular beacons from the nucleic acid that comprises the nucleotide sequence of interest. The figure also depicts related compositions.

### DETAILED DESCRIPTION

### Overview

The present invention is generally directed to compositions, methods, systems, and kits that can be useful for determining the nucleotide sequence of a nucleic acid. In general, sequencing a nucleic acid according to the invention entails annealing labeled hybridization probes to a single-stranded nucleic acid of interest. In certain embodiments, this can include hybridizing the probes to one strand of a double-stranded nucleic acid that has been made single stranded by, e.g., denaturation, enzymatic digestion of one strand, or other available methods, e.g., those described in U.S. patent application Ser. No. 12/383,855 and U.S. patent application Ser. No. 12/286,119. Probes are then sequentially removed from the nucleic acid by, e.g., an enzyme or enzyme complex that exhibits probe-displacing activity, e.g., a helicase, a polymerase, a ribosome, or the like. A sequence of transient signals is produced by the step-wise removal of the probes from the nucleic acid.

The signals are then detected and converted into nucleotide sequence information, thus providing the sequence of the nucleic acid of interest.

The sequencing methods provided herein can circumvent the need for costly, labor-intensive nucleic amplification and labeling, which can limit sequencing template sample production from matching the capacities of modern sequencing systems (such systems are reviewed in, e.g., Chan et al. (2005) “Advances in Sequencing Technology” Mutation Research 573: 13-40, and described in Levene et al. (2003) “Zero Mode Waveguides for Single Molecule Analysis at High Concentrations,” Science 299: 682-686). Furthermore, the methods and compositions of the invention can be cost-effectively multiplexed in order to increase throughput.

The detailed description is organized to first elaborate the various methods and compositions provided by the invention for determining the nucleotide sequence of a nucleic acid. Next, details regarding probe-displacing enzymes, labeled hybridization probes, and sequencing systems into which the compositions and methods of the invention can be integrated are described. Broadly applicable molecular biological techniques that can be used with the invention are described thereafter.

### Methods and Compositions for Sequencing a Nucleic Acid Using Labeled Hybrization Probes and Probe-Displacing Enzymes

The methods and compositions provided by the invention can be used to determine the nucleotide sequence, e.g., a single-stranded nucleic acid or one strand of a double-stranded nucleic acid that has been made single stranded by, e.g., denaturation, enzymatic digestion of one strand, or other available methods, e.g., those described in U.S. patent application Ser. No. 12/383,855 and U.S. patent application Ser. No. 12/286,119. As used herein, a “nucleotide sequence” is the consecutive order of covalently linked nucleotides in a nucleic acid. Unlike current sequencing-by-synthesis (SBS) or sequencing-by-hybridization (SBH) strategies, the methods provided herein advantageously permit the direct sequencing of a nucleic acid, e.g., without requiring time-consuming, expensive amplification steps and/or chemical labeling steps. In the methods, one or more labeled hybridization probes is annealed to a template nucleic acid. The probe(s) are then dissociated from the template with an enzyme that exhibits probe-displacing activity to produce a transient signal. The signal, or a sequence of signals, is detected and converted into nucleotide sequence information, thus determining the sequence of the template nucleic acid.

Nucleic acids that can be sequenced using the invention include, but are not limited to, e.g., oligonucleotides, cDNAs, genomic DNAs, and RNAs. Alternatively or additionally, a nucleic acid sequenced using the methods provided herein can comprise a string of nucleotides, which string represents the nucleotide sequence of interest, e.g., a DNA, an RNA, or the like. Nucleic acids sequenced according to the methods of the invention can optionally comprise nucleotide analogs, labeled nucleotides, and/or the like. In addition, nucleic acids can optionally be produced synthetically or prepared or isolated from any of a variety of sources, including, e.g., eukaryotes, mammals, prokaryotes, viruses, and others, as described elsewhere herein.

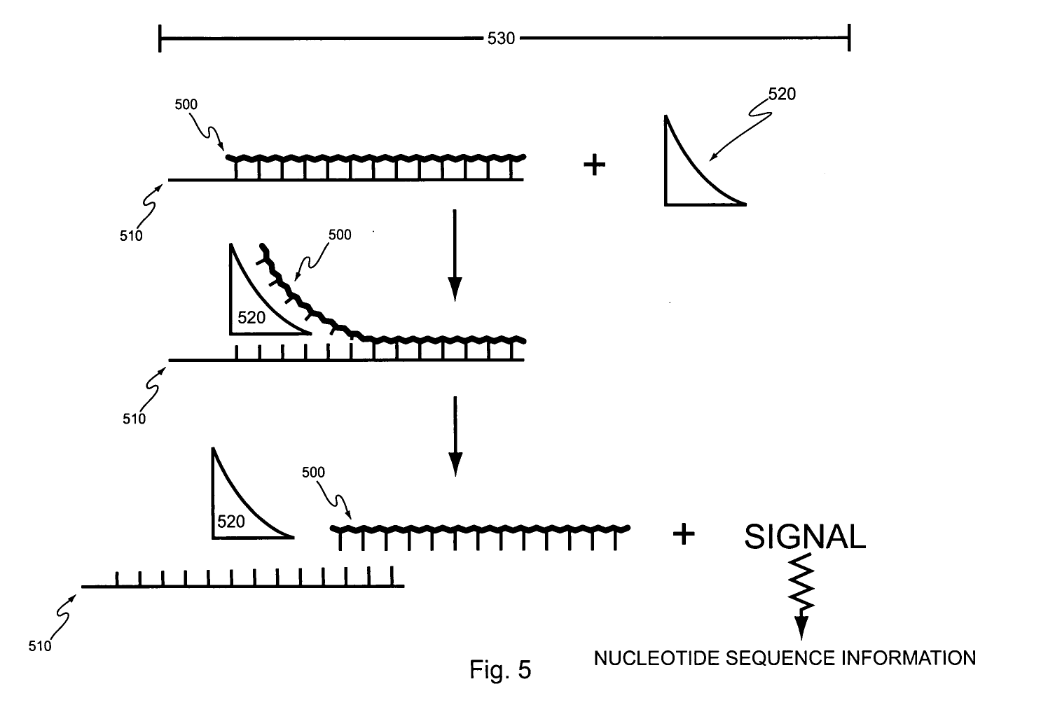

General methods and compositions provided by the invention are schematically illustrated in FIG. 5. In a first step, nucleic acid 510 is provided and hybridized to labeled hybridization probe 500. In a next step, probe 500 is displaced from nucleic acid 510 by probe-displacing enzyme 520. The removal of probe 500 from nucleic acid 510 produces a signal, e.g., an optical signal, that is then detected, e.g., by a detection module, and converted, e.g., by a conversion module, into nucleotide sequence information, e.g., the nucleotide subsequence of nucleic acid 510 to which probe 500 was hybridized. In some embodiments, the nucleic acid in compositions provided by the invention, e.g., composition 530, comprises a sequence of interest, e.g., the sequence that is to be determined by methods provided herein. In such embodiments, the labeled probes hybridized to the nucleic acid can each comprise a short complementary subsequence, e.g., less than 3 nucleotides, 3-16 nucleotides, or more than 16 nucleotides, of the sequence of interest. In general, any oligonucleotide probe that comprises a label, e.g., a fluorescent label, a magnetic label, a quantum dot, a gold nanoparticle, or the like, that produces a detectable signal upon the removal of the probe from the nucleic acid to which is hybridized can be used in the methods.

In certain embodiments, the optically labeled hybridization probes present in the compositions provided by the invention are molecular beacons. As used herein, a “molecular beacon” refers to a single-stranded oligonucleotide hybridization probe that comprises a self-complementary sequence capable of forming a stem-loop structure in solution, and which typically comprises a covalently linked fluorophore at one end and a covalently linked quencher at the second end. A “quencher” is a moiety that alters a property of, e.g., a fluorescent label, when it is in proximity to the label. The quencher can actually quench an emission, but it does not have to, i.e., it can simply alter some detectable property of the label, or, when proximal to the label, cause a different detectable property than when not proximal to the label. A quencher can be, e.g., an acceptor fluorophore that operates via energy transfer and re-emits the transferred energy as light. Other similar quenchers, e.g., dark quenchers such as Dabsyl, Iowa black FQ, Iowa black RQ, and others, do not re-emit transferred energy as light. Dark quenchers return to their ground states via nonradiative or dark decay, wherein dissipated energy is given off via molecular vibrations (heat). Further details regarding molecular beacons are described in U.S. Pat. No. 5,925,517 (Jul. 20, 1999) to Tyagi et al., entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits”, which are incorporated by reference in their entireties. In preferred embodiments of the methods and compositions herein, a molecular beacon's optical label not detectable by an optical detection module when the label is quenched.

In the compositions provided herein, the loop of each molecular beacon comprises a sequence that is complementary to a subsequence of a nucleic acid of interest, e.g., whose nucleotide sequence is to be determined using the methods herein. Hybridizing molecular beacons to the nucleic acid of interest forces disassociation of the molecular beacons' stems, thereby distancing the fluorophores and quenchers from each other.

Typically, dissociation of a molecular beacon's stems unquenches the fluorophore, causing an increase in fluorescence of the molecular beacon. However, in preferred embodiments of the compositions, molecular beacons are hybridized to a nucleic acid of interest in a head-to-tail arrangement. As used herein, a “head-to-tail arrangement” refers to an arrangement of molecular beacons hybridized to a single-stranded nucleic acid wherein the molecular beacons abut one another along the nucleic acid, and wherein the fluorophore, or “head” of each molecular beacon is proximal to the quencher, or “tail”, of the preceding molecular beacon, e.g., the molecular beacon that is hybridized to an adjacent upstream subsequence.

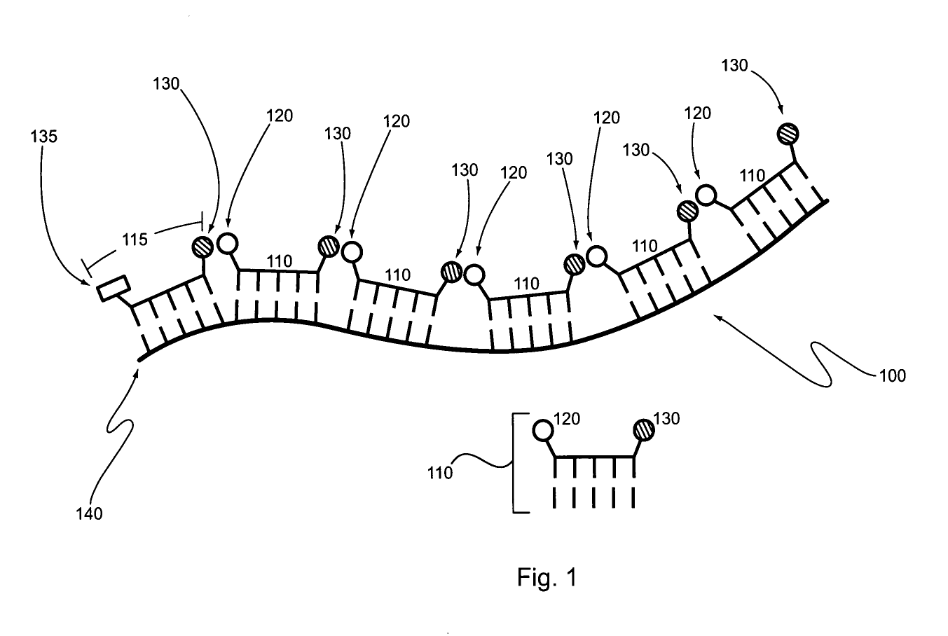

A schematic depiction of a “head-to-tail arrangement” of molecular beacons on a single-stranded nucleic acid is shown in FIG. 1. Starting at first end 140 of single-stranded nucleic acid 100, molecular beacons 110 are hybridized to adjacent complementary subsequences on nucleic acid 100. The fluorophore “heads” 120 of each of the molecular beacons are proximal to first end 140 of nucleic acid 100, and quencher “tails” 130 of each molecular beacon abut the fluorophore “head” of the neighboring downstream molecular beacon. Molecular beacon 115, which comprises fluorophore “head” 135 is hybridized to a subsequence at first end 140 of nucleic acid 100, and does not abut a quencher “tail”. Thus, fluorophore 135 will fluoresce.

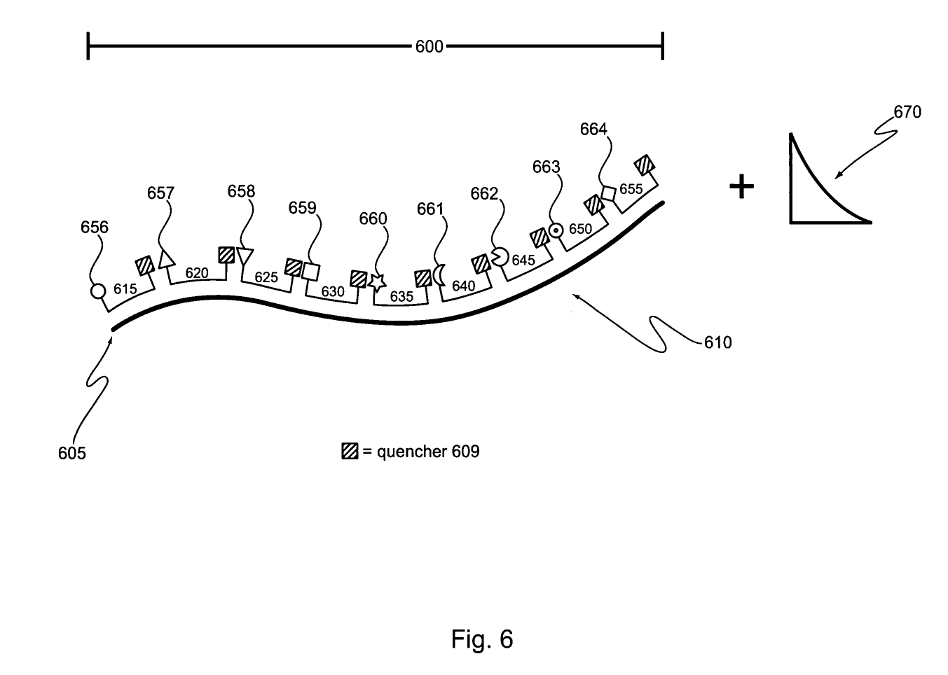

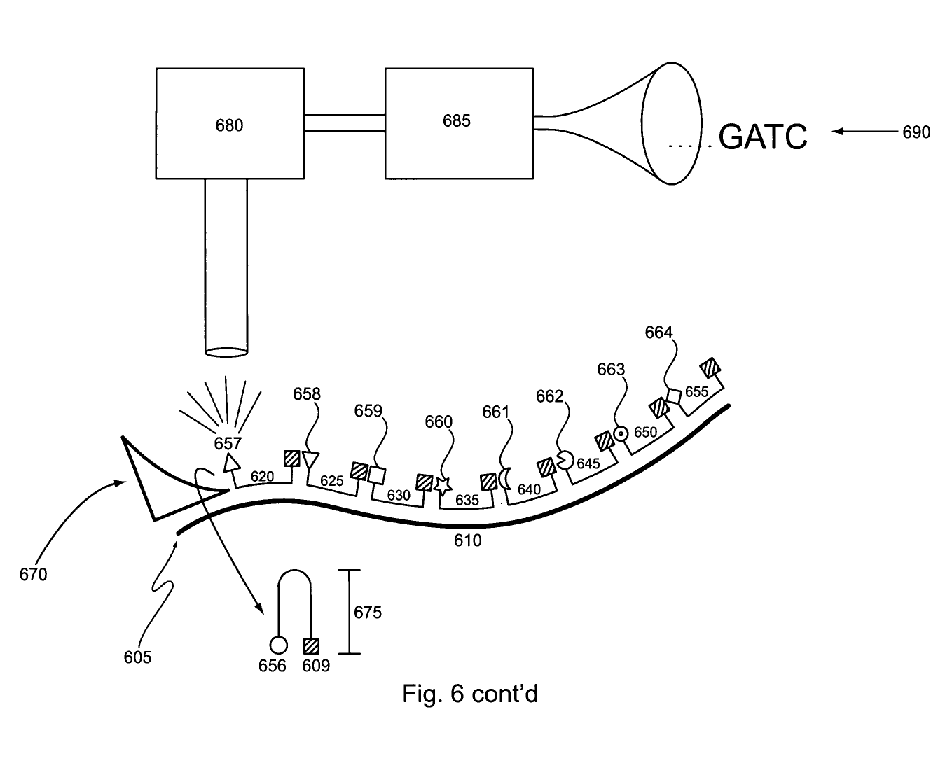

The nucleotide sequence of a nucleic acid interest is be determined by the removal, e.g., consecutive removal, of molecular beacons that are hybridized to a nucleic acid in a “head-to-tail” arrangement. For example, as shown in FIG. 6, composition 600 includes nucleic acid 610, e.g., an RNA, a DNA, an oligonucleotide, or the like. Composition 600 also includes a series of molecular beacons, e.g., molecular beacons 615 655, that are annealed to nucleic acid 610. Molecular beacons 615-655 each comprise quencher 609 at one end. The loops of molecular beacons 615-655 each comprise a short, e.g., four nucleotide long, loop sequence. Each loop sequence is complementary to a unique, e.g., four nucleotide long, subsequence of nucleic acid 610, e.g., starting at first end 605. Each molecular beacon also comprises a fluorophore, e.g., one of fluorophores 656-664, which fluorophore corresponds to the molecular beacon's particular four nucleotide loop sequence. In other words, every four nucleotide subsequence of nucleic acid 610, e.g., starting from first end 605, is hybridized to a molecular beacon that comprises a fluorophore whose fluorescent signal corresponds to that unique four nucleotide subsequence. One of skill in the art will immediately recognize, however, that a molecular beacon's loop sequence, and the subsequence of the nucleic acid to which the molecular beacon's loop sequence hybridizes, need not be limited to a length of four nucleotides. Beneficially, each fluorophore that corresponds to a unique, e.g., four nucleotide, subsequence in nucleic acid 610, emits a fluorescent signal that is distinguishable, e.g., by an optical detection module, from the signals produced by the other fluorophores in the composition.

As a result of their head-to-tail arrangement, fluorophores 657-664 (see FIG. 6) of molecular beacons 620-655, e.g., hybridized to nucleic acid 610, abut quenchers 609 of the molecular beacons hybridized to an upstream subsequence of nucleic acid 610. Consequently, the molecular beacons do not produce a fluorescent signal when hybridized to nucleic acid 610 in this arrangement, thus beneficially reducing undesired background fluorescence. However, as shown in FIG. 6, molecular beacon 615, which is hybridized to a nucleotide subsequence at first end 605 of single-stranded nucleic acid 610, does not abut the quencher “tail” of an upstream molecular beacon. Thus, fluorophore 656 will fluoresce. Because the signal produced by fluorophore 656 of molecular beacon 610 corresponds to a particular four nucleotide subsequence at first end 605 of nucleic acid 610, the first four nucleotides of nucleic acid 610 can be determined from fluorophore 656's fluorescent signal.

Generally, a probe-displacing enzyme (e.g., a DNA helicase, an RNA helicase, an RNA/DNA helicase, a DNA polymerase, an RNA polymerase, a reverse transcriptase, or the like) present in the compositions provided herein sequentially removes the one or more labeled hybridization probes that is annealed to the nucleic acid of interest to produce a signal, e.g., an optical signal, that can be converted into nucleotide sequence information. As shown in FIG. 6, a probe-displacing enzyme, e.g., probe-displacing enzyme 670, is introduced to composition 600. Probe displacing enzyme 670, can displace, e.g., consecutively displace, molecular beacons 615-655 from nucleic acid 610, e.g., starting at first end 605 of nucleic acid 610. However, one of skill in the art will recognize that the removal of optically labeled probes, e.g., molecular beacons 615-655, from a nucleic acid, e.g., nucleic acid 600, by a probe-displacing enzyme, e.g., probe-displacing enzyme 670, need not necessarily start from first end 605. For example, the sequential removal of molecular beacons 615-655 from nucleic acid 610 by probe-displacing enzyme 670 can optionally begin from either end of nucleic acid 610, e.g., depending on the directionality of probe-displacing 670. Optionally, probe-displacing enzyme 670 can begin removing molecular beacons at an internal site on nucleic acid 610 and proceed in either direction, depending on the enzyme's directionality.

As each successive molecular beacon is “unzipped” from nucleic acid 610 by probe-displacing enzyme 670, the molecular beacon will form a stem-loop structure, e.g., stem-loop structure 675, that brings the molecular beacon's fluorophore, e.g., fluorophore 656, into proximity with its quencher, e.g., quencher 609, thus preventing its own fluorescence. (Example molecular beacons are provided and described in, e.g., U.S. Pat. No. 5,925,517 (Jul. 20, 1999) to Tyagi et al., entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”) The fluorophore of the next downstream molecular beacon, e.g., fluorophore 657, will no longer abut a quencher and can fluoresce, emitting an optical signal that is detected by signal detector module 680. Detector module 680 then transmits fluorescent signal information to conversion module 685. Because, as noted above, each unique fluorescent signal corresponds to a unique four nucleotide subsequence in the nucleic acid of interest, the conversion module can convert the sequence of fluorescent signals into nucleotide sequence information, e.g., nucleotide sequence 690. Nucleotide sequence information can be transmitted one or more output devices, such as a printer and/or a monitor to display results, and the like. Thus, sequence of fluorescent signals e.g., produced by the displacement of molecular beacons from a nucleic acid by a probe-displacing enzyme, corresponds to the sequence of the nucleic acid from which the molecular beacons were “unzipped”.

In other embodiments, the nucleic acid present in compositions provided by the invention, e.g., the nucleic acid to which molecular beacons are annealed and subsequently “unzipped”, comprises a coded representation of a sequence of interest, e.g., wherein each nucleotide in the sequence of interest is represented, e.g., in the nucleic acid of the composition, by, e.g., one or more unique oligonucleotides. For example, preferred embodiments of compositions described herein include a single-stranded DNA concatamer that comprises a unique sequence of two code units. As used herein, “code units” refer to oligonucleotide segments, e.g., less than 10 nucleotides long, approximately 10 nucleotides long, or more than 10 nucleotides long, that can be used to represent the nucleotide sequence of, e.g., a DNA of interest, an RNA of interest, or the like.

The sequence of a nucleic acid of interest can optionally be converted into a format wherein each nucleotide in the sequence is encoded as a sequence of code units, e.g., two binary code units. Converting a the nucleotide sequence of a nucleic acid of interest into a sequence of, e.g., binary code units, can beneficially simplify the readout process because the identities of the two code units, rather than those of four nucleotides, need to be resolved by a signal detector. For example, if a first code unit represents the binary digit “0” and a second code unit represents the binary digit “1”, then each of the four nucleotides in a template nucleic acid, e.g., a DNA or an RNA, can be substituted with a particular combination of two code units, e.g., 00, 01, 10, or 11, such that each nucleotide is encoded by a two-bit binary value. In preferred embodiments, converting the nucleotide sequence of a nucleic acid of interest into a string of binary code values, e.g., that represent the nucleotide sequence of the nucleic acid of interest, does not entail a prior knowledge of the nucleotide sequence of the nucleic acid of interest. For example, such conversion methods and kits are described in, e.g., U.S. Pat. No. 6,723,513 B2, by Lexow et al., entitled, “SEQUENCING METHOD USING MAGNIFYING TAGS” issued Apr. 20, 2004.

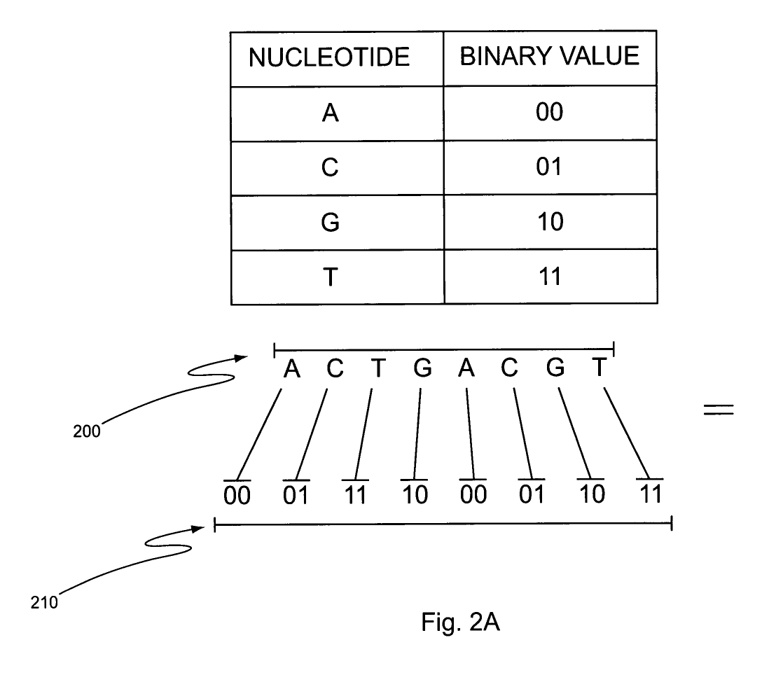

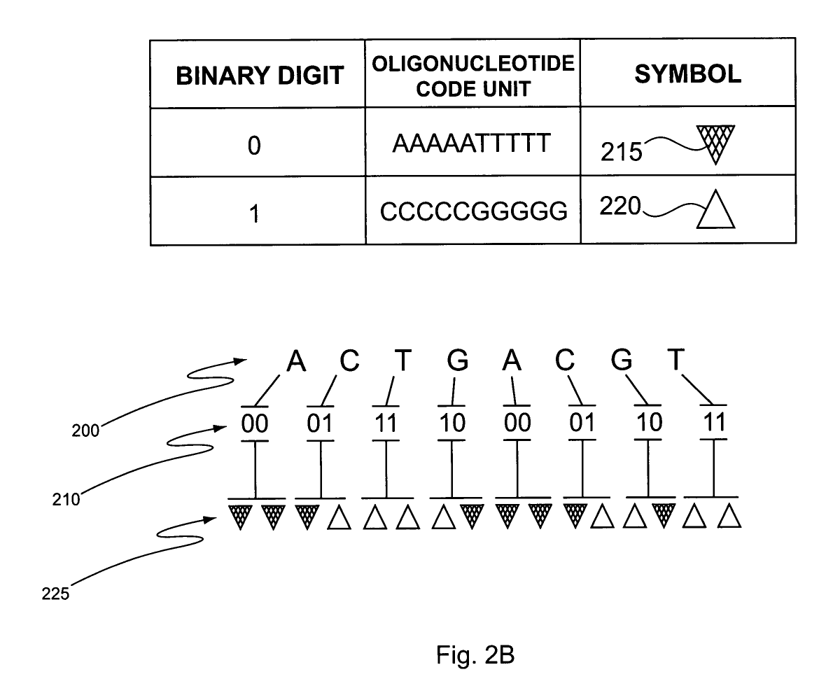

For example, as shown in FIG. 2A, template nucleic acid 200 comprises the sequence ACTGACGT. If A=00, C=01, G=10, and T=11, then the nucleotide sequence of template nucleic acid 200 can be represented in binary code as binary string 210, or 0001111000011011. (One of skill in the art will immediately recognize that nucleotides A, C, G, and T can be represented by any of the four two-digit binary values 00, 01, 10, and 11, and that the assignments above need not be taken as limiting.) Binary string 210 can then be converted into a sequence of covalently linked code units, wherein first code unit 215, which represents the binary digit “0”, comprises, e.g., a nucleotide 10-mer with the sequence, e.g., aaaaattttt; and second code unit 220, which represents the binary digit “1”, comprises, e.g., a nucleotide 10-mer with the sequence, e.g., cccccggggg (see FIG. 2B). In preferred embodiments of the invention, the sequences of each covalently linked code unit are chosen to minimize the formation of secondary structures in the resulting concatamer of code units. Methods and kits that can be used to convert a nucleic acid into a sequence of binary code unit concatamers are described in further detail in U.S. Pat. No. 6,723,513 B2, by Lexow et al., entitled, “SEQUENCING METHOD USING MAGNIFYING TAGS” issued Apr. 20, 2004, and are available from Ling Vitae (Norway). Nucleic acids comprising up to 25 nucleotides, up to 40 nucleotides, or, more preferably, up to 100 nucleotides can be converted into such concatamers (Soni et al. (2007) “Progress toward Ultrafast DNA Sequencing Using Solid-State Nanopores” Clin Chem 53: 1996-2001).

It will be apparent to one of skill in the art that the code units used with the invention need not be limited to the sequences and/or the lengths of the code units described above. Code units can be, e.g., less than 5 nucleotides long, 5-10 nucleotides long, or more than 10 nucleotides long. In addition, one of skill in the art will appreciate that each nucleotide in e.g., a DNA of interest, an RNA of interest, or the like, e.g., sequenced according to the methods provided by the invention, need not be represented in binary code format. For example, each nucleotide in, e.g., a nucleic acid of interest that is to be sequenced according to the methods herein, can be represented in trinary code format, quadnary code format, or other formats.

By substituting the appropriate code units for the digits in binary string 210, single-stranded DNA concatamer 225, which is schematically depicted in FIG. 2 with symbols that represent first code units 215 and second code units 220, can be produced. As will be discussed further below, the conversion of the nucleotide sequence of a template nucleic acid, e.g., nucleic acid 200, to a concatamer comprising a sequence of code unit pairs, e.g., concatamer 225, can simplify the readout process because the identities of the two code units, rather than those of four nucleotides, need to be resolved by a signal detector.

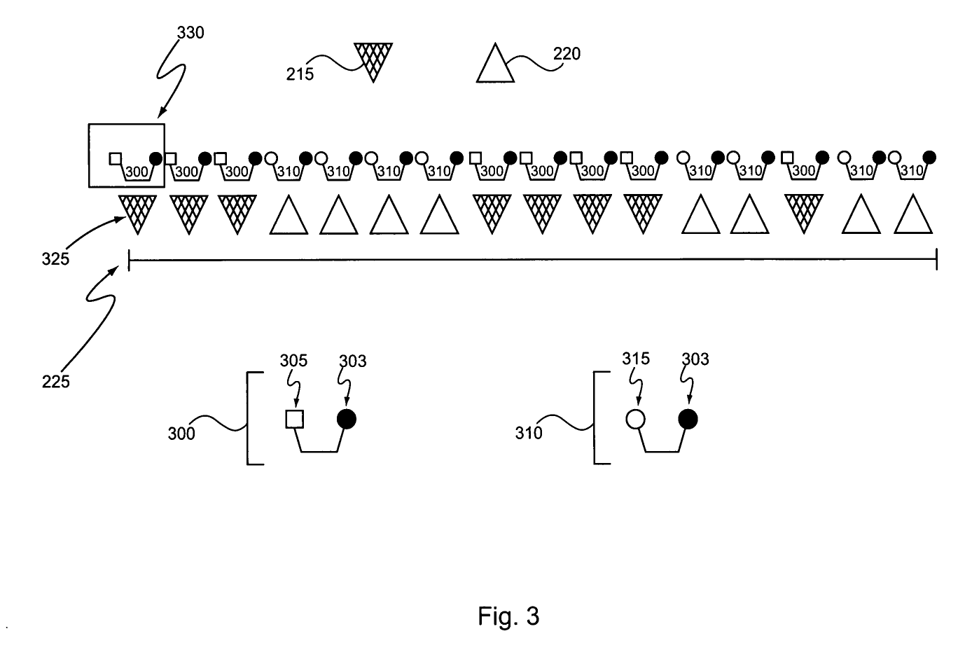

A nucleotide sequence can be determined by the sequential removal of molecular beacons from, e.g., a concatamer of code units, e.g., concatamer 225 (see FIG. 3). In FIG. 3, code units 215 are represented in concatamer 225 by shaded triangles, and code units 220 are represented in concatamer 225 by open triangles (see FIG. 2 and corresponding description for detailed explanation). As noted previously, concatamer 225 is a string of binary values that represents the nucleotide sequence of nucleic acid 200 (see FIG. 2 and corresponding description for detailed explanation). First molecular beacons 300, which each comprise a loop sequence complementary to code units 215, and second molecular beacons 310, which each comprise a loop sequence complementary to code units 220, can be hybridized to concatamer 225. First molecular beacons 300 each additionally comprise covalently linked first fluorophore 305 at one end and covalently linked quencher 303 at the second end. Second molecular beacons 310 each comprise covalently linked second fluorophore 315 at one end and covalently linked quencher 303 at the second end. Fluorophores 305 and 315 each emit light at a unique wavelength that is readily distinguishable, e.g., by an optical signal detector, e.g., optical signal detector 425, from the wavelength of light emitted by the other fluorophore. The sequence of fluorescent signals produced by the removal of molecular beacons from concatamer 225 provides the information that will be converted, e.g., by conversion module 430, into the nucleotide sequence of the nucleic acid of interest, e.g., the nucleotide sequence of nucleic acid 200, which is represented by the sequence of code units in concatamer 225.

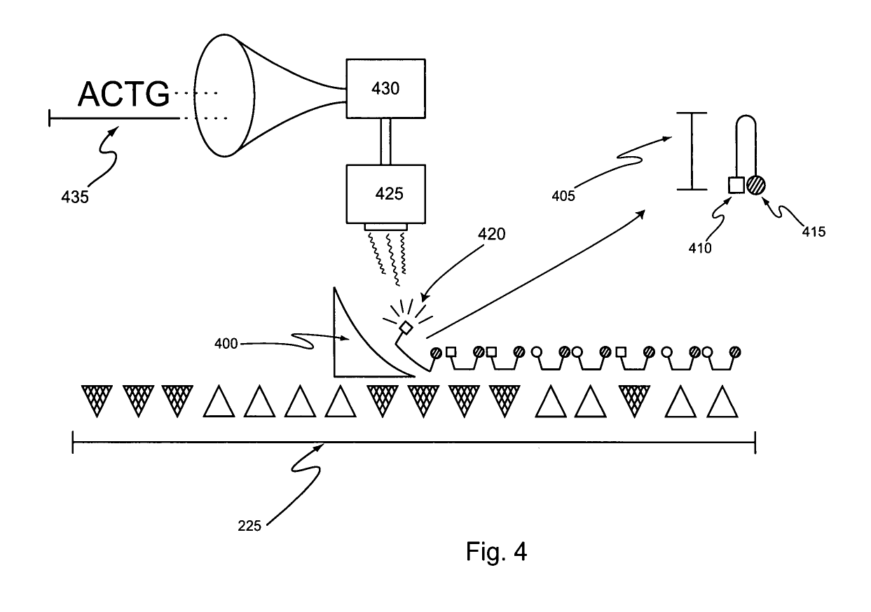

As depicted in FIG. 4, a probe-displacing enzyme, e.g., helicase 400, is introduced to concatamer 225, to which first molecular beacons 300 and second molecular beacons 310 are hybridized. Helicase 400 sequentially displaces molecular beacons 300 and 310 from concatamer 225, e.g., starting at the first end 325. As described previously, one of skill in the art will recognize that the removal of labeled probes need not necessarily start from an end, e.g., first end 325, of a concatamer of code units to which molecular beacons have been annealed. When each successive molecular beacon is fully “unzipped” from concatamer 225 by helicase 400 and released into solution, it will form a stem-loop structure, e.g., stem loop structure 405, which brings its fluorophore, e.g., fluorophore 410, into proximity of its quencher, e.g., quencher 415, preventing its own fluorescence. The fluorophore of the next downstream molecular beacon, e.g., molecular beacon 420, fluoresces, emitting a signal that is detected by optical signal detector 425, which transmits fluorescent signal information to conversion module 430, which converts the sequence of transient fluorescent signals into nucleotide sequence information 435. The sequence information can then be transmitted to one or more output devices, such as a printer and/or a monitor to display results, and the like.

Thus, in the embodiments, a sequence of fluorescent signals e.g., produced by the displacement of molecular beacons from a nucleic acid by a probe-displacing enzyme, corresponds to a coded representation of the nucleotide sequence of a nucleic acid of interest.

In certain embodiments, the detection systems described herein distinguish single-bit signals, e.g., two states, e.g., “0” or “1”, rather than two-bit information, e.g., four states, e.g., “A”, “C”, “G” or “T”. Thus, if A=00, C=01, G=10, and T=11, detection systems can be configured to detect one of two fluorescent signals. The signal information can then be transmitted to a conversion module, which is configured to convert each consecutive pair of fluorescent signals into nucleotide information. As noted previously, one of skill in the art will immediately recognize that nucleotides A, C, G, and T can be represented by any of the four two-digit binary values 00, 01, 10, and 11; and that the assignments above need not be taken as limiting.

Advantageously, the sequences of the “0” and “1” code units, and, accordingly, of the molecular beacons that hybridize to the code units, can be engineered to maximize the contrast between the oligonucleotide sequence of each code unit to minimize cross hybridization of, e.g., a “0” molecular beacon to a “1” code unit and vice versa, thus simplifying the conversion of signals to nucleic acid sequence data and reducing the error rate in determining the nucleic acid sequence. Furthermore, the conversion of, e.g., at least 10² different single-stranded nucleic acid templates, at least 10³ different single-stranded nucleic acid templates, or, most beneficially, at least 10⁴ different single-stranded nucleic acid templates into DNA concatamers of code unit pairs can be performed in parallel, maximizing sample production (See, e.g., U.S. Pat. No. 6,723,513 B2, by Lexow et al., entitled, “SEQUENCING METHOD USING MAGNIFYING TAGS” issued Apr. 20, 2004).

Any of the compositions described herein can optionally be present, e.g., on a planar surface, in a well, in an observation volume, or in a single-molecule reaction region, such as a ZMW. In certain embodiments, probe-displacing enzymes are immobilized on a solid support, e.g., a glass cover slip, a planar surface, a well, an observation volume, or a single-molecule reaction region, thus localizing the fluorescent signal to enhance detection and readout. Surface attachment of a probe-displacing enzyme can be particularly advantageous in multiplexing the methods of the invention, e.g., to increase sequencing throughput. For example, a population of probe-displacing enzymes can optionally be arranged on a solid support in a micro patterned array, or they can be randomly localized. Optionally, probe-displacing enzymes can each be localized to single wells of a ZMW.

Certain embodiments of the invention include determining the sequence of a nucleic acid comprising more than 4 unique kinds of nucleotide. One of average skill in the art will readily recognize that each nucleotide in such a nucleic acid can be most beneficially represented, e.g., in a concatamer of code units, in a code unit format other than a binary code format. Such alternative code unit formats can include, e.g., trinary code, wherein each nucleotide is represented by three code units; quadnary code, wherein each nucleotide is represented by four code units, or others.

The methods described herein can optionally be performed in, e.g., a thermocycler, and the strand displacing enzyme used to remove molecular beacons from the nucleic acid to which they are hybridized can be, e.g., a thermostable Taq polymerase. For example, the template nucleic acid can be sequenced according to the methods described above, e.g., via the Taq-mediated removal of molecular beacons that are hybridized to the template nucleic acid. The temperature of the reaction mix present in the thermocycler can then be increased to remove any molecular beacons that remain bound to the template nucleic acid and to release the nucleic acid from the Taq. The temperature of the reaction mix can then be lowered to permit the re-hybridization of the molecular beacons to the template and to allow the Taq polymerase to rebind the template. The enzyme can then “re-displace” the molecular beacons from the template, and the sequence of signals produced by their removal can be detected and converted into nucleotide sequence information, e.g., by the appropriate system modules. The reaction mix can optionally include an excess of molecular beacons to permit efficient rehybridization. The thermostable Taq can optionally be surface-bound, and the template nucleic acid can optionally be, e.g., a single-stranded closed loop.

Methods of sequencing using molecular beacons are described in Deamer et al. (2000) “Nanopores and nucleic acids: prospects for ultrarapid sequencing” Trends Biotechnol 18:147-51. However, in the present invention, molecular beacons are “unzipped” from a concatamer of code units by probe-displacing enzymes, rather than by translocation through a nano-scale pore. Using the methods of the invention, the approximate number of molecular beacons that are displaced by a probe-displacing enzyme in a given amount of time can be calculated, and this calculation can greatly facilitate consistent read-out and minimize the possibility that the detection of an “unzipping event” is missed. Furthermore, the compositions of the present invention can be more cost-effective and more easily scaled for high-throughput than those described in Soni et al. (2007) “Progress toward Ultrafast DNA Sequencing Using Solid-State Nanopores” Clin Chem 53: 1996-2001. In addition, because probe-displacing enzymes can optionally be arranged in a micro patterned arrays, detection systems can be advantageously localized to enhance fluorescence detection and readout. Such detection systems can also be multiplexed to monitor the removal of molecular beacons from, e.g., at least 10,000 unique concatamers of unit code pairs, at least 100,000 unique concatamers of unit code pairs, or at least 1,000,000 unique concatamers of unit code pairs in parallel.

### Further Details Regarding Probe-Displacing Enzymes

Methods for determining the order of nucleotides in a nucleic acid have significantly accelerated biological research and discovery. Currently, nucleic acid sequence data are valuable in myriad applications in biological research and molecular medicine, including determining the hereditary factors in disease, in developing new methods to detect disease and to guide therapy (van de Vijver et al. (2002) “A gene-expression signature as a predictor of survival in breast cancer,” New England Journal of Medicine 347: 1999-2009), in drug development, and in providing a rational basis for personalized medicine. The present invention is directed to methods and compositions useful for sequencing a nucleic acid. In the methods, labeled hybridization probes, e.g., molecular beacons are annealed to a nucleic acid of interest or a concatamer, e.g., of code units, representative thereof. Each probe is then removed, e.g., sequentially, from the nucleic acid by an enzyme or enzyme complex that exhibits probe-displacing activity.

Helicases are one example of probe-displacing enzymes that can be used in the methods provided herein. Helicases are a class of NTP-dependent motor proteins that play a critical role in every aspect of RNA and DNA metabolism, e.g., DNA replication, DNA repair, transcription, recombination, translation, ribosome biogenesis, RNA splicing, etc. Helicases typically move directionally along the phosphodiester backbone of the nucleic acid to which they are bound, using the energy produced by nucleic acid-dependent NTP hydrolysis to translocate along the nucleic acid while catalyzing the separation of two strands of a complementary nucleic acid duplex, e.g., two annealed DNA strands, two annealed RNA strands, a DNA strand annealed to an RNA strand, etc. In preferred embodiments of the invention, helicases used in the methods can include, e.g., a uvrD, a Rep, a RecQ, a dnaB, a T4 gp41, or a T7 gp4.

Structural studies of many diverse helicases have shown that all helicases studied to date comprise the Walker A and Walker B motifs, whose most conserved residues are implicated in nucleotide binding and hydrolysis (reviewed in Gorbalenya et al. (1993) “Helicases: amino acid sequence comparisons and structure-function relationships.”Curr Opin Struct Biol 3: 419-429). In addition, all helicases whose structures are known contain a core fold that was first visualized in the crystal structure of RecA (Bailey et al. “The crystal structure of the Thermus aquaticus DnaB helicase monomer.” Nucl Acids Res 35: 4728-4736). Specific helicase families and superfamilies, e.g., Superfamily I, which includes UvrD and Rep; Superfamily II, which includes RecQ; Superfamily III, which includes E1 and Adenovirus Rep; the DnaB-like family, which includes dnaB, T4 gp41, and T7 gp4; and the Rho-like family, which includes Rho; are defined by the presence of additional specific motifs. Helicases within a family will typically share similar three-dimensional folds (Subramanya et al. (1996) “Crystal structure of a DExx box DNA helicase.” Nature 384: 379-383; Bird et al. (1998) “Helicases: a unifying structural theme?”Curr Opin Struct Biol 8: 14-18; Subramanya et al. (1996) “Crystal Structure of an ATP-Dependent DNA Ligase from Bacteriophage T7.” Cell 85: 607-615; Singleton et al. (2000) “Crystal structure of T7 gene 4 ring helicase indicates a mechanism for sequential hydrolysis of nucleotides.” Cell 101: 589-600). However, despite their structural similarity, helicases within a family or superfamily can exhibit different substrate specificities, e.g., DNA, RNA or both DNA and RNA, directionality, e.g., 5′→3′ vs. 3′→5′; and different processivities.

Even though helicases share similar structural folds, they can assemble into a variety of oligomeric forms, e.g., ranging from monomers to hexamers. Typically, the functionally active forms of many multi-subunit helicases are oligomeric. For example, the monomers of ring-shaped hexameric helicases such as, e.g., E. coli DnaB and Rho, T4 gp41, and T7 gp4, cannot hydrolyze NTPs or catalyze the unwinding of duplex DNA. The helicase activity of many homodimeric and/or heterodimeric helicases, e.g., UvrD and RecBCD, respectively, is greatly enhanced by the formation of dimers. Many monomeric helicases, e.g., T4 Dda, exhibit functional cooperativity and enhanced processivity when loaded onto the same strand of a duplex nucleic acid, despite the fact that they do not form stable oligomers nor show cooperativity in NTP hydrolysis or nucleic acid binding.

Most helicases require a single-stranded nucleic acid region from which to initiate strand separation. In general, helicases bind to single-stranded nucleic acids with higher affinity than to double-stranded nucleic acids, and this binding is sequence independent. Hexameric ring-shaped helicases, such as those mentioned previously, often require Y-shaped nucleic acid structures with a loading strand of an optimum length to initiate unwinding (Jewsewska et al. (1997) “Complex of Escherichia coli Primary Replicative Helicase DnaB Protein with a Replication Fork: Recognition and Structure.” Biochemistry 37: 3116-3136; Matson et al. (1983) “The gene 4 protein of bacteriophage T7. Characterization of helicase activity.” J Biol Chem 258: 14017-14024; Venkatesan et al. (1982) “Bacteriophage T4 gene 41 protein, required for the synthesis of RNA primers, is also a DNA helicase.” J Bio Chem 257: 12426-12434). Nevertheless, certain helicases, e.g., RecBCD, SV40 Large T, and RuvB can bind to double-stranded DNA and initiate unwinding from blunt-ended duplex DNA. Once loaded onto a nucleic acid strand, most helicases exhibit a directional bias, e.g., 5′→3′ vs. 3′→5′. Helicases can exhibit varying degrees of tolerance to changes in the loading strand during translocation. For example, some helicases are sensitive to breaks in the nucleic acid, electrostatic disruptions, or abasic sites (Eoff et al. (2005) “Chemically Modified DNA Substrates Implicate the Importance of Electrostatic Interactions for DNA Unwinding by Dda Helicase.” Biochemistry 44: 666-674), whereas others, e.g., T4 Dda, show no sensitivity to disruptions in the loading strand (Tackett et al. (2001) “Unwinding of Unnatural Substrates by a DNA Helicase.” Biochemistry 40: 543-548).

The translocation and base pair separation activities of helicases are driven by NTP binding and hydrolysis, wherein the NTP hydrolysis cycle is hypothesized to be coupled with a conformational change that produces, e.g., a “power stroke” (Jiang et al. (1994) “Mechanics of myosin motor: force and step size.” Bioessays 16: 531-532) or “Brownian ratchet” (Astunian (1997) “Thermodynamics and kinetics of a Brownian motor.” Science 276: 917-922) that propels the enzyme along the loading strand while destabilizing a nucleic acid duplex. These mechanisms are discussed in further detail in, e.g., Gaur (2006) “Helicase: Mystery of progression.” Molec Biol Reports 34: 161-164; Lee et al. (2006) “UvrD Helicase Unwinds DNA One Base Pair at a Time by a Two-Part Power Stroke.” Cell 127: 1349-1360; Rasnik et al. (2008) “Branch migration enzyme as a Brownian ratchet.” EMBO J. 27: 1727-35; and “Helicases as Molecular Motors.” In Schliwa ed. Molecular Motors (pp 179-203) Hoboken, N.J.: Wiley-VCH.

As used herein, the “kinetic step size” of a helicase is defined as the number of base pairs unwound between observed two successive rate limiting lags in the unwinding of nucleic acid duplexes of various lengths by a helicase, e.g., in vitro. The kinetic step sizes of many helicases have been experimentally determined. For example, a kinetic step size of 3-4 base pairs has been reported for, e.g., UvrD (Ali et al. (1997) “Kinetic Measurement of the Step Size of DNA Unwinding by Escherichia coli UvrD Helicase.” Science 275: 377-380), and a kinetic step sizes of 9-10 base pairs has been reported for, e.g., T7 gp4 and DnaB (Jeong et al. (2004) “The DNA-unwinding mechanism of the ring helicase of bacteriophage T7.” Proc Natl Acad Sci USA 101: 7264-7269; Galletto et al. (2004) “Unzipping mechanism of the double-stranded DNA unwinding by a hexameric helicase: Quantitative analysis of the rate of the dsDNA unwinding, processivity, and kinetic step-size of the E. coli DnaB helicase.” J Mol Biol 343: 83-99).

A helicase that exhibits a kinetic step size smaller than, e.g., the length of a code unit, can complete several kinetic steps before it removes a molecular beacon from the code unit to which it is hybridized, e.g., see FIG. 4 and corresponding description. Though the duration of each of the helicase's kinetic steps can vary, the average duration of a kinetic step can be used to approximate the number of molecular beacons that are expected to be displaced in a given amount of time. This calculation can greatly facilitate consistent read-out and minimize the possibility that the detection of an “unzipping event” is missed. For example, if a helicase has a kinetic step size equivalent to that of the length of one code unit, and the duration of kinetic step is known, one can predict the average number of fluorescent signals that are to be detected by, e.g., a detection module of a sequencing system. This information can be useful, e.g., in optimizing reactions to perform the sequencing methods described herein.

Further details regarding helicase translocation mechanisms; helicase base pair separation mechanisms; and/or assays to measure helicase translocation rate, processivity or step size are elaborated in, e.g., Singleton et al. (2007) “Structure and Mechanism of Helicases and Nucleic Acid Translocases.” Ann Rev Biochem 76: 23-50; Pyle (2008) “Translocation and Unwinding Mechanisms of RNA and DNA Helicases.” Ann Rev Biophys 37: 317-333; Tuteja et al. (2004), “Prokaryotic and eukaryotic DNA helicases: Essential molecular motor proteins for cellular machinery.” Eur J Biochem 271: 1835-1848; Bleichert et al. (2007) “The long and unwinding road of RNA helicases.” Mol Cell 27: 339-52; and Levin and Patel (2003) “Helicases as Molecular Motors.” In Schliwa, ed. Molecular Motors (pp 179-203) Hoboken, N.J.: Wiley-VCH.

In certain embodiments, a multi-protein complex such as a ribosome can be used to displace molecular beacons from, e.g., an RNA template to which they have been hybridized. Ribosomes are complexes of RNA and protein that are found in all cells. Each ribosome comprises two subunits, a 30S subunit and a 50S subunit that together form an 80S complex to translate mRNA into a polypeptide chain. Details regarding the structure and activities of the ribosome are elaborated in, e.g., Ramakrishnan (2002) “Ribosome Structure and the Mechanism of Translation.” Cell 108: 557-572; Laurberg et al. (2008) “Structural basis for translation termination on the 70S ribosome.” Nature doi:10.1038/nature07115; Wen et al. (2008) “Following translation by single ribosomes one codon at a time.” Nature 452: 598-603; and Noller, H F (2006) “Biochemical characterization of the ribosomal decoding site.” Biochimie 88: 932-41.

In other embodiments of the methods, other probe-displacing enzymes, e.g., RNA polymerases, DNA polymerases, and/or reverse transcriptases, along with any additional necessary accessory proteins, can be used to displace labeled hybridization probes annealed to a nucleic acid, e.g., a nucleic acid comprising a sequence of interest or a nucleic acid that comprises a coded representation of a sequence of interest. For example, a concatamer that comprises a promoter upstream of, e.g., the sequence code units on a DNA concatamer, can be produced such that an RNA polymerase can be used to displace, e.g., molecular beacons that have been hybridized to the concatamer, during transcription. Similarly, a nucleic acid comprising a primer hybridization site upstream of the sites to which hybridization probes anneal can be produced, and a DNA polymerase, e.g., a T4 or T7 DNA polymerase, can displace the probes during replication. In another embodiment, a reverse transcriptase could displace, probes hybridized to an RNA during reverse transcription.

DNA polymerases that can be used in methods of the invention and in related compositions are generally available. DNA polymerases are sometimes classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol Chem 276: 43487-90. For a review of polymerases, see, e.g., Hübscher et al. (2002) “Eukaryotic DNA Polymerases” Annual Review of Biochemistry 71: 133-163; Alba (2001) “Protein Family Review: Replicative DNA Polymerases” Genome Biology 2:reviews 3002.1-3002.4; and Steitz (1999) “DNA polymerases: structural diversity and common mechanisms” J Biol Chem 274: 17395-17398. The basic mechanisms of action for many polymerases have been determined. The sequences of literally hundreds of polymerases are publicly available.

As described above, an RNA polymerase can also be used to displace probes hybridized to a nucleic acid, e.g., by transcribing the template to which labeled probes are hybridized. Whereas single subunit RNA polymerases are found in some bacteriophages, mitochondria, some eukaryotic organelles, multi-subunit RNA polymerases can be found in bacteria, archaea, and eukaryotes. Although they share no apparent sequence or structural homology, most RNA polymerases carry out the basic steps of transcription in an identical manner. To initiate synthesis, an RNA polymerase binds to a specific promoter sequence in the DNA template that lies upstream of the start site for transcription. The enzyme then separates (melts) the two strands of the template near the start signal to form a transcription “bubble”, and begins RNA synthesis using the coding strand of the downstream DNA as a template and a single ribonucleotide as a primer, displacing the second DNA strand. E. coli RNA polymerase has been reported to exhibit a kinetic step size of 1 nucleotide (Abbondanzieri et al. (2005) “Direct observation of base-pair stepping by RNA polymerase.” Nature 438: 460-4650). The average duration of an RNA polymerase kinetic step size can be used to estimate the amount of time in which the polymerase can displace a molecular beacon of a given length from the nucleic acid to which it is hybridized, as described above.

Reverse transcriptases also comprise probe-displacing activity. Reverse transcriptase enzymes possess an RNA-dependent DNA polymerase activity and a DNA-dependent DNA polymerase activity, both of which can be useful in displacing probes that are hybridized to a nucleic acid. Though most reverse transcriptases perform the same fundamental activities, they differ with respect to their processivities, the optimal temperatures and pHs at which they exhibit activity, etc. Though reverse transcriptases do not share any significant sequence homology with DNA polymerases, the structures of HIV RT and E. coli Klenow share significant characteristic features, indicating that their polymerization mechanisms may be similar.

Additional details regarding DNA polymerases, RNA polymerases, and/or reverse transcriptases are discussed in, e.g., Johnson et al. (2005) “Cellular DNA replicases: components and dynamics at the replication fork.” Annu Rev Biochem 74: 283-315; Cramer (2004) “Structure and Function of RNA polymerase II.” Adv Protein Chem 67: 1-42; Trinh et al. (2006) “Structural perspective on mutations affecting the function of multisubunit RNA polymerases.” Micro Mol Bio Rev 70: 12-36; Cheetham (2000) “Insights into transcription: structure and function of single-subunit DNA-dependent RNA polymerases.” Curr Opin Struct Biol 10: 117-123; Borukhov et al. (2008) “RNA polymerase: the vehicle of transcription.” Trends Microbiol 16: 126-134; and Mullard (2008) “Reverse transcription: do the flip.” Nat Rev Molec Cell Biol 6: 500-510. Kits for transcription, DNA amplification, and reverse transcription are detailed below.

Additional Details Regarding Labeled Hybridization Probes and Methods of their Detection

The methods provided by the invention entail dissociating labeled hybridization probes from a nucleic acid template, e.g., in a sequential manner, with a probe-displacing enzyme to produce a signal. In general, any oligonucleotide probe that comprises a label, e.g., a fluorescent label, a magnetic label, a quantum dot, a gold nanoparticle, or the like, that produces a detectable signal upon the removal of the probe from the nucleic acid to which is hybridized can be used in the methods.

In preferred embodiments of the compositions provided herein, a set of molecular beacons, e.g., that are hybridized to, e.g., a single-stranded template nucleic acid or a double-stranded nucleic acid that has been made single-stranded via, e.g., denaturation, enzymatic digestion of one strand, or other available methods, e.g., those described in U.S. patent application Ser. No. 12/383,855 and U.S. patent application Ser. No. 12/286,119, can be sequentially removed to produce a sequence of transient signals, e.g., fluorescent signals, that can be detected and converted into sequence information. Alternatively, the molecular beacons can be annealed to a nucleic acid that comprises a coded representation of a nucleotide sequence of interest, e.g., a concatamer of code units. (See, e.g., Soni et al. (2007) “Progress toward Ultrafast DNA Sequencing Using Solid-State Nanopores” Clin Chem 53: 1996-2001.) As described above, a molecular beacon is a single-stranded oligonucleotide hybridization probe that comprises a self-complementary sequence capable of forming a stem-loop structure. Each “arm” of the stem-forming sequences of a molecular beacon is typically 5-7 nucleotides long, but one of skill in the art will recognize that the lengths and/or the complementary sequences of a molecular beacon's arms need not be limiting.

When a molecular beacon is present free in solution, i.e., not hybridized to a second nucleic acid, the stem of the molecular beacon is stabilized by complementary base pairing. This self-complementary pairing results in the formation of the stem-loop, wherein the fluorophore and the quenching moieties are proximal to one another. In this confirmation, the fluorescent moiety is quenched by the fluorophore. In the compositions of the invention, the loops of the molecular beacons comprise sequences that are complementary to subsequences in the nucleic acid of interest, e.g., whose sequence is to be determined using the methods described herein. Thus, hybridization of the loop sequence of a molecular beacon to its complementary subsequence in the nucleic acid of interest forces disassociation of the stem, thereby distancing the fluorophore and quencher from each other.

Typically, dissociation of a molecular beacon's stems unquenches the fluorophore, causing an increase in fluorescence of the molecular beacon. However, in preferred embodiments of the compositions provided herein, molecular beacons are preferably hybridized to a nucleic acid of interest, e.g., whose nucleotide sequence is to be determined using the methods herein, in “head-to-tail” arrangement. This configuration prevents the fluorescence of the molecular beacons, e.g., wherein the fluorophore “heads” of each molecular beacon is proximal to the quencher “tail” of the molecular beacon hybridized to the code unit directly upstream (see FIG. 1 and corresponding description). This arrangement advantageously reduces undesired background fluorescence, thus increasing the signal strength during the strand displacing enzyme-assisted “unzipping” of the beacons from the concatamer. As noted previously, one molecular beacon annealed to an end of a nucleic acid will not abut an upstream quencher and will fluoresce (see, e.g., molecular beacons 115 and 615, hybridized to nucleotide subsequences at the first ends 140 and 605 of single-stranded nucleotide polymers 100 and 610, in FIGS. 1 and 6, respectively). Molecular beacons are described in further detail in references cited hereinbelow, which are incorporated by reference in their entireties.

The fluorescent signal produced by, e.g., the dissociation of a probe, e.g., a molecular beacon, from a nucleotide polymer, can be detected by any of a number of techniques well known in the art, e.g., a donor-quencher interaction, multicolor fluorescence detection, FRET, Total Internal Reflection Fluorescence (TIRF), etc. See, e.g., Geddes and Lakowicz, eds. Reviews in Fluorescence (2006) Hoboken: Springer-Verlag; Suhling et al. (2005) “Time-resolved fluorescence microscopy.” Photochem Photobiol Sci 4: 13-22; Dietrich et al. (2002) “Fluorescence resonance energy transfer (FRET) and competing processes in donor-acceptor substituted DNA strands: a comparative study of ensemble and single-molecule data.” J Biotechnol 82: 211-31; and other references below.

In certain embodiments of the compositions, the molecular beacons hybridize, e.g., in a head-to-tail configuration, to a nucleic acid that comprises the nucleotide sequence of interest that is to be determined using the methods described herein. Thus, in such embodiments, the molecular beacons each comprise a sequence that is complementary to a subsequence of the nucleic acid. In other embodiments of the compositions, the molecular beacons hybridize, e.g., in a head-to-tail configuration, to a nucleic acid that comprises a coded representation of a nucleic acid sequence of interest, e.g., wherein each nucleotide in the sequence of interest is represented, e.g., in the nucleic acid of the composition, by, e.g., one or more code units (see FIGS. 2-4 and the corresponding description for a detailed explanation). In such compositions, the molecular beacons comprise sequences that are complementary to each code unit in the nucleic acid to which they are hybridized.

Using the methods of the invention, the sequence of a nucleic acid can be determined by the sequential removal of, e.g., fluorescently labeled oligonucleotide probes, from the nucleic acid to which they are hybridized by a strand displacing enzyme, e.g., a helicase, a DNA polymerase, and RNA polymerase, a ribosome, or a reverse transcriptase. The probes' removal from the nucleic acid can produce a signal that can be detected via fluorescence polarization measurements, which can provide information regarding the probes' molecular orientations and mobility. The binding of a fluorescently labeled hybridization probe to a concatamer of code units significantly decreases the amount of rotation of the labeled probe/concatamer complex over that of the free probe. This has a corresponding effect on the level of polarization that is detectable. Specifically, the probe, when hybridized to a concatamer, exhibits a much higher fluorescence polarization than the unbound, labeled probe. See, e.g., United States Patent Application Publication No. 20040166553. Decreases in the probe's fluorescence polarization, e.g., following the removal of the probe from the concatamer by a helicase, can be detected and converted into nucleotide sequence information.

Fluorescent labels can be introduced to oligonucleotides during synthesis or by post synthetic reactions by techniques established in the art; for example, kits for fluorescently labeling polynucleotides with various fluorophores are available from Molecular Probes, Inc. ((www.) molecularprobes.com), and fluorophore-containing phosphoramidites for use in nucleic acid synthesis are commercially available. Quantum dots can also be covalently linked to a nucleic acid (Zhou et al., (2008) “A compact functional quantum Dot-DNA conjugate: preparation, hybridization, and specific label-free DNA detection.” Langmuir 24: 1659-1664). Similarly, signals from the labels (e.g., absorption by and/or fluorescent emission from a fluorescent label) can be detected by essentially any method known in the art. As described above, multicolor detection, detection of FRET, TIRF, fluorescence polarization, and the like, are well known in the art.

Molecular beacons or other probes can be custom synthesized, e.g., by Alta Bioscience (United Kingdom), Biosearch Technologies (Novato, Calif.), TriLink BioTechnologies (San Diego, Calif.), Thermo Fisher Scientific (Massachusetts), and others. Fluorophores that are most commonly linked to molecular beacons include fluorescien, HEX, TET, Cy5 or Cy3, Coumarin, Texas Red, and Tamra, although a molecular beacon can be synthesized to comprise any one of a variety of fluorophores and/or fluorophore and quencher combinations. For example, gold-quenched molecular beacons exhibit a high quenching efficiency (Dubertret et al. (2001) “Single-mismatch detection using gold-quenched fluorescent oligonucleotides.” Nat Biotechnol 19: 365-70). Quantum dot-conjugated molecular beacons are also available (Kim et al. (2007) “Multicolour hybrid nanoprobes of molecular beacon conjugated quantum dots: FRET and gel electrophoresis assisted target DNA detection.” Nanotechnology 18: 195105-195111). The folding of the designed sequence of a custom molecular beacon can be modeled with available software, see, e.g., Monroe et al. (2003) “Molecular beacon sequence design algorithm.” Biotechniques 34: 68-70, 72-73; and AlleleID®, available from Premiere Biosoft International, can indicate whether the intended stem-and-loop conformation can occur.

Further details regarding the synthesis, and use of molecular beacons is described in, e.g., Leone et al. (1995) “Molecular beacons: probes that fluoresce upon hybridization” Nature Biotechnology 14: 303-308; Blok and Kramer (1997) “Amplifiable hybridization probes containing a molecular switch” Mol Cell Probes 11: 187-194; Hsuih et al. (1997) “Novel, ligation-dependent PCR assay for detection of hepatitis C in serum” J Clin Microbiol 34: 501-507; Kostrikis et al. (1998) “Molecular beacons: spectral genotyping of human alleles” Science 279: 1228-1229; Sokol et al. (1998) “Real time detection of DNA:RNA hybridization in living cells” Proc Natl Acad Sci USA 95: 11538-11543; Tyagi et al. (1998) “Multicolor molecular beacons for allele discrimination” Nature Biotechnology 16: 49-53; Bonnet et al. (1999) “Thermodynamic basis of the chemical specificity of structured DNA probes” Proc Natl Acad Sci USA 96: 6171-6176; Fang et al. (1999) “Designing a novel molecular beacon for surface-immobilized DNA hybridization studies” J Am Chem Soc 121: 2921-2922; Marras et al. (1999) “Multiplex detection of single-nucleotide variation using molecular beacons” Genet Anal Biomol Eng 14: 151-156; Vet et al. (1999) “Multiplex detection of four pathogenic retroviruses using molecular beacons” Proc Natl Acad Sci USA 96: 6394-6399; U.S. Pat. No. 5,925,517 (Jul. 20, 1999) to Tyagi et al., entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

### Further Details Regarding Systems

The methods and compositions provided by the invention can advantageously be integrated with systems that can, e.g., automate and/or multiplex the probe-displacing enzyme-assisted removal of labeled hybridization probes from a nucleic acid, e.g., to determine the nucleotide sequence of a nucleic acid. Systems of the invention can include one or more modules, e.g., that automate a method herein, e.g., for high-throughput sequencing applications. Such systems can include fluid-handling elements and controllers that move reaction components into contacts with one another, signal detectors, system software/instructions, e.g., to convert a sequence of fluorescent signals into nucleotide sequence information, and the like.

Systems provided by the invention can include a reaction region in which one or more probe-displacing enzyme, e.g., a helicase, can remove, e.g., sequentially remove, labeled probes that have been annealed to a nucleic acid. The reaction region can optionally comprise a planar surface, e.g., a glass cover slip, e.g., on which one or more probe-displacing enzyme has been immobilized, e.g., using any one or more methods well known to one of skill in the art. For example, a population of probe-displacing enzymes can optionally be arranged on a solid support in a micro patterned array, or they can be randomly localized. Alternatively or additionally, reaction region can optionally comprise one or more well, a single-molecule reaction region, or observation volume, e.g., a ZMW. In a preferred embodiment, probes can simultaneously be displaced from up to 1,000, up to 10,000, or up to 100,000 templates in a reaction region of a system of the invention to increase sequencing throughput.

Systems of the invention can optionally include modules that provide for detection or tracking of products. Detectors can include spectrophotometers, CCD arrays, CMOS arrays, microscopes, cameras, or the like. Optical labeling is particularly useful because of the sensitivity and ease of detection of these labels, as well as their relative handling safety, and the ease of integration with available detection systems (e.g., using microscopes, cameras, photomultipliers, CCD arrays, CMOS arrays and/or combinations thereof). High-throughput analysis systems using optical labels include DNA sequencers, array readout systems, cell analysis and sorting systems, and the like. For a brief overview of fluorescent products and technologies see, e.g., Sullivan (ed) (2007) Fluorescent Proteins, Volume 85, Second Edition (Methods in Cell Biology) (Methods in Cell Biology) ISBN-10: 0123725585; H of et al. (eds) (2005) Fluorescence Spectroscopy in Biology: Advanced Methods and their Applications to Membranes, Proteins, DNA, and Cells (Springer Series on Fluorescence) ISBN-10: 354022338X; Haughland (2005) Handbook of Fluorescent Probes and Research Products, 10th Edition (Invitrogen, Inc./Molecular Probes); BioProbes Handbook, (2002) from Molecular Probes, Inc.; and Valeur (2001) Molecular Fluorescence: Principles and Applications Wiley ISBN-10: 352729919X.

System software, e.g., instructions running on a computer can be used to track and inventory reactants or products, and/or for controlling robotics/fluid handlers to achieve transfer between system stations/modules. Systems provided by the invention will beneficially include a conversion module that assembles the signals, e.g., fluorescent signals produced by the removal of labeled hybridization probes from a template, into an overall sequence of a nucleic acid, e.g., the nucleic acid from which the probes are being removed. Systems that can be adapted to the invention are generally described in Soni et al. (2007) “Progress toward Ultrafast DNA Sequencing Using Solid-State Nanopores” Clin Chem 53: 1996-2001; U.S. Pat. No. 6,723,513 B2, to Lexow et al., entitled, “SEQUENCING METHOD USING MAGNIFYING TAGS” issued Apr. 20, 2004; and PCT/US 0865996, filed Jun. 5, 2008 by Tomaney et al., entitled, “METHODS AND PROCESSES FOR CALLING BASES IN SEQUENCE BY INCORPORATION METHODS”. A conversion module can include additional software and instructions for its use. The overall system can optionally be integrated into a single apparatus, or can consist of multiple apparatus with overall system software/instructions providing an operable linkage between modules.

### Further Details Regarding Broadly Used Molecular Biology Techniques

Preparing Nucleic Acid Samples

Methods for determining the order of nucleotides in a nucleic acid have significantly accelerated biological research and discovery. Currently, nucleic acid sequence data are valuable in myriad applications in biological research and molecular medicine, including determining the hereditary factors in disease, in developing new methods to detect disease and to guide therapy (van de Vijver et al. (2002) “A gene-expression signature as a predictor of survival in breast cancer,” New England Journal of Medicine 347: 1999-2009), in drug development, and in providing a rational basis for personalized medicine. The methods provided by the invention can be used to determine the sequence of a nucleotide polymer, which in certain embodiments, can include, e.g., a DNA fragment derived from a genomic DNA, an mRNA, cDNA, and the like. Though DNA concatamers of code units are used in some embodiments of the methods, Samples comprising a population of, e.g., DNAs, RNAs, mRNAs, or cDNAs, can be prepared using techniques that are well known in the art.

Preparing Genomic DNA

Genomic DNA can be prepared from any source, e.g., eukaryotic, prokaryotic, archaeal, viral, etc., by three steps: cell lysis, deproteinization and recovery of DNA. These steps are adapted to the demands of the application, the requested yield, purity and molecular weight of the DNA, and the amount and history of the source. Further details regarding the isolation of genomic DNA can be found in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2008 (“Sambrook”); Current Protocols in Molecular Biology, F. M. Ausubel et al., eds., Current Protocols, a joint venture between Greene Publishing Associates, Inc. and John Wiley & Sons, Inc (“Ausubel”); Kaufman et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley). In addition, many kits are commercially available for the purification of genomic DNA from cells, including Wizard™ Genomic DNA Purification Kit, available from Promega; Aqua Pure™ Genomic DNA Isolation Kit, available from BioRad; Easy-DNA™ Kit, available from Invitrogen; and DnEasy™ Tissue Kit, which is available from Qiagen.

Preparing RNA and cDNA

Alternative splicing (AS) is a major source of protein diversity in higher eukaryotic organisms, and this process is frequently regulated in a developmental stage-specific or tissue-specific manner. Thus, an understanding of changes in splicing patterns can be critical to a comprehensive understanding of biological regulation and disease. Nucleic acid sequence data obtained from sequencing cDNAs, according to the methods of the invention, can be useful in identifying novel splice variants of a gene of interest and/or in comparing the differential expression of splice isoforms of a gene of interest, e.g., between different tissue types, between different treatments to the same tissue type or between different developmental stages of the same tissue type. cDNAs are prepared from mRNA. mRNA can typically be isolated from almost any source using protocols and methods described in, e.g., Sambrook and Ausubel. The yield and quality of the isolated mRNA can depend on, e.g., how a tissue is stored prior to RNA extraction, the means by which the tissue is disrupted during RNA extraction, or on the type of tissue from which the RNA is extracted. RNA isolation protocols can be optimized accordingly. Many mRNA isolation kits are commercially available, e.g., the mRNA-ONLY™ Prokaryotic mRNA Isolation Kit and the mRNA-ONLY™ Eukaryotic mRNA Isolation Kit (Epicentre Biotechnologies), the FastTrack 2.0 mRNA Isolation Kit (Invitrogen), and the Easy-mRNA Kit (BioChain). In addition, mRNA from various sources, e.g., bovine, mouse, and human, and tissues, e.g., brain, blood, and heart, is commercially available from, e.g., BioChain (Hayward, Calif.), Ambion (Austin, Tex.), and Clontech (Mountainview, Calif.).

Once the purified mRNA is recovered, reverse transcriptase is used to generate cDNAs from the mRNA templates. Methods and protocols for the production of cDNA from mRNAs, e.g., harvested from prokaryotes as well as eukaryotes, are elaborated in cDNA Library Protocols, I. G. Cowell, et al., eds., Humana Press, New Jersey, 1997, Sambrook and Ausubel. In addition, many kits are commercially available for the preparation of cDNA, including the Cells-to-cDNA™ II Kit (Ambion), the RETROscript™ Kit (Ambion), the CloneMiner™ cDNA Library Construction Kit (Invitrogen), and the Universal RiboClone® cDNA Synthesis System (Promega). Many companies, e.g., Agencourt Bioscience and Clontech, offer cDNA synthesis services.

Preparing DNA Concatamers

Short sequence tags can be linked together to from long serial molecules termed “concatamers” that can be sequenced, e.g., using the methods described herein. A short sequence tag, e.g., 10-14 bp, can contain sufficient information to uniquely identify a transcript, provided that that the tag is obtained from a unique sequence within the transcript. Quantitation of the number of times a particular tag is observed provides the expression level of the corresponding transcript. Thus, sequencing the nucleic acid templates, e.g., according to the methods provided by the invention, derived from concatenated short ESTs, e.g., in a high-throughput sequencing system, can be useful in analyzing global gene expression patterns of, e.g., a tissue at different developmental stages, tissues in different organs from a common genotype, common tissues of different genotypes, common tissues that have been exposed to different treatments, and the like. In addition, sequencing templates, e.g., produced using method described herein, derived from concatamers of short ESTs can eliminate the need for a practitioner to carry out laborious and time-consuming in vivo cloning and cell culturing techniques that are common for other EST-based systems for the analysis of global gene expression, e.g., SAGE (Velculescu et al. (1995) “Serial analysis of gene expression.” Science 270: 484-487) and TALEST (Spinella et al (1999) “Tandem arrays ligation of expressed sequence tags (TALEST): a new method for generating global gene expression profiles.” Nucl Acid Res 27: e22).

Preparing concatenated ESTs can comprise preparing a cDNA library, e.g., as described above. Typically, the prepared cDNA can then be digested with a restriction enzyme that would be expected to cleave most transcripts at least once, e.g., a restriction enzyme with a 4-base pair recognition site. The 3′-most cDNA fragments are then captured and ligated to adapter molecules that each contain a type-II restriction site, e.g., BsgI, and a second restriction site. Digestion of the adapter-ligated cDNAs, e.g., with BsgI, produces DNA fragments that consist of the adapter itself and an additional 10-12 nucleotides of unknown cDNA sequence separated from the adapter by the restriction site originally used to digest the cDNA. The fragments can then be ligated to a second adapter containing a second restriction site at one end and degenerate overhangs, e.g., which render the second adapter compatible with all possible cDNA sequences, e.g., produced by the BsgI digestion, at the other. The resulting double-tagged DNA molecules can be digested with enzymes that recognize the restriction sites on the adapters and ligated together to form concatamers that can then be prepared, e.g., using the methods described herein, for sequencing, e.g., using a high-throughput system. Additional information and methods describing the preparation of concatamers comprising short ESTs can be found in, e.g., Velculescu et al. (1995) “Serial analysis of gene expression.” Science 270: 484-487; Spinella et al (1999) “Tandem arrays ligation of expressed sequence tags (TALEST): a new method for generating global gene expression profiles.” Nucl Acid Res 27: e22; WIPO Patent Application Number WO/2004/024953; and Unneberg et al. (2003) “Transcript identification by analysis of short sequence tags—influence of tag length, restriction site, and transcript database.” Nucl Acids Res 31: 2217-2226.

Converting a Nucleic Acid Sequence into a String of Binary Values

Rather than directly reading the sequence of a nucleic acid template, preferred embodiments of methods described herein, e.g., of sequencing a nucleic acid, use a single-stranded DNA concatamer that comprises a unique sequence of two binary code units, e.g., code units that represent binary digits 0 and 1, such that the sequence of unit codes in the concatamer represents the sequence of nucleotides in the original nucleic acid template (see FIG. 2 and corresponding description). The use of such concatamers in the methods can be beneficial in simplifying the readout process, in that only two distinguishable fluorescent signals, rather than four, e.g., one for each nucleotide, need to be detected by a fluorescence detection system.

In one method of synthesizing the concatamers, a nucleic acid that is to be sequenced, e.g., a DNA, can be fragmented to produce a population of nucleic acid fragments ≦1 kb in length. DNA adapter tags that include an MmeI recognition site are then ligated to the fragments. The tagged fragments are then digested with Mmel, a type II restriction enzyme that cuts 20 base pairs into the sequence of each fragment and leaves a 2-base pair overhang. Second adapter tags that include a SfaNI recognition site are then ligated to the overhangs generated by the MmeI digestion. Following these steps, a conversion cycle wherein three bases are removed, e.g., via SfaNI digestion, from one end of each fragment, e.g., DNA fragment, in the population, and six corresponding code units are ligated to the second end. The selection of only the “correct” DNA adapter is performed by PCR amplification in each cycle (“Rapid DNA Sequencing by Direct Nanoscale Reading of Nucleotide Bases on Individual DNA chains.” In Mitchelson, ed. New High Throughput Technologies For DNA Sequencing and Genomics (pp 245-261) Amsterdam, The Netherlands: Elsevier). Advantageously, this cyclic process has been optimized to be highly parallel such that, e.g., up to 100, up to 1000, or up to 10,000 different nucleic acid fragments can be converted into code unit concatamers is a single test tube. Further details regarding methods for the conversion of nucleic acids into concatamers of code units can be found in U.S. Pat. No. 6,723,513 B2, to Lexow et al., entitled, “SEQUENCING METHOD USING MAGNIFYING TAGS” issued Apr. 20, 2004, the entirety of which has been incorporated herein by reference. In addition, kits for the conversion of DNA fragments into code unit concatamers are available from LingVitae (Norway).

Generating Nucleic Acid Fragments

The methods of preparing single-stranded nucleic acids that are described herein can entail generating fragments from, e.g., a genomic DNA, a cDNA, or a DNA concatamer. Double-stranded nucleic acid fragments are then made single-stranded, e.g., via denaturation, enzymatic digestion of one strand, or other available methods, and molecular beacons, which comprise sequences that are complementary to subsequences present on the single-stranded fragment, are annealed to the fragment in a “head-to-tail” arrangement. There exist a plethora of ways of generating nucleic acid fragments from a genomic DNA, a cDNA, or a DNA concatamer. These include, but are not limited to, mechanical methods, such as sonication, mechanical shearing, nebulization, hydroshearing, and the like; enzymatic methods, such as exonuclease digestion, restriction endonuclease digestion, and the like; and electrochemical cleavage. These methods are further explicated in Sambrook and Ausubel.

Amplification of Template Nucleic Acids

The most widely used in vitro technique among these methods is polymerase chain reaction (PCR), which requires the addition of a template of interest, e.g., a DNA comprising the sequence that is to be amplified, nucleotides, oligonucleotide primers, buffer, and an appropriate polymerase to an amplification reaction mix. In PCR, the primers anneal to complementary sequences on denatured template DNA and are extended with a thermostable DNA polymerase to copy the sequence of interest. As a result, a nucleic acid that comprises a sequence complementary to that of the template strand (or “target strand”) is synthesized. Repeated cycles of PCR can generate myriad copies. Primers ideally comprise sequences that are complementary to the template. However, they can also comprise sequences that are not complementary, but which comprise e.g., restriction sites, cis regulatory sites, oligonucleotide hybridization sites, protein binding sites, DNA promoters, RNA promoters, sample or library identification sequences, and the like. Primers can comprise modified nucleotides, such as methylated, biotinylated, or fluorinated nucleotides; and nucleotide analogs, such as dye-labeled nucleotides, non-hydrolysable nucleotides, and nucleotides comprising heavy atoms. Primers can be custom synthesized by commercial suppliers as described below. PCR can be a useful means by which to attach tags to fragments. Further details regarding PCR and its uses are described in PCR Protocols A Guide to Methods and Applications (Innis et al. eds) Academic Press Inc. San Diego, Calif. (1990) (Innis); Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

Additional methods that can be used to amplify, or copy, nucleic acids include strand displacement amplification (SDA), multiple-displacement amplification (MDA), rolling circle replication (RCR). Some methods use RCR to copy single-stranded nucleic acids, e.g., which will be used as templates in sequencing reactions, from double-stranded templates. In RCR, DNA replication is initiated by an initiator protein, e.g., cis A, which nicks one strand of the double-stranded, closed DNA loop at a specific nucleotide sequence called the double-strand origin, or DSO. The initiator protein remains bound to the 5′ phosphate end of the nicked strand, and the free 3′ hydroxyl end is released to serve as a primer for DNA synthesis by DNA polymerase III. Using the un-nicked strand as a template, replication proceeds around the DNA loop, displacing the nicked strand as single-stranded DNA. Displacement of the nicked strand is carried out by a replisome, e.g., a multiprotein complex that comprises a single-stranded DNA binding protein (SSB), a helicase, a polymerase, and an RCR initiation protein, e.g., cisA.

Further details regarding Rolling Circle Amplification can be found in Demidov et al. (2002) “Rolling-circle amplification in DNA diagnostics: the power of simplicity,” Expert Rev Mol Diagn 2: 89-94; Demidov and Broude (eds) (2005) DNA Amplification: Current Technologies and Applications. Horizon Bioscience, Wymondham, UK; and Bakht et al. (2005) “Ligation-mediated rolling-circle amplification-based approaches to single nucleotide polymorphism detection” Expert Rev Mol Diagn 5: 111-116; Koonin et al. (1993) “Computer-assisted dissection of rolling circle DNA replication.”BioSystems 30: 241-268; and Novick (1998) “Contrasting Lifestyles of rolling-circle phages and plasmids.” TIBS 23: 434-438.

Copying steps in the methods can also be a method by which single-stranded nucleic acids can be produced, e.g., for sequencing using the methods described herein. Such copying steps can be performed with a strand-displacing polymerase. The term “strand displacement” describes the ability of a polymerase to displace downstream DNA encountered during synthesis. Examples of strand-displacing polymerases that can be used with the methods include, e.g., a Phi29 polymerase, a Poll polymerase, a BstI polymerase, or a Phi29-like polymerases, such as those described in U.S. patent application Ser. No. 11/645,223, entitled POLYMERASES FOR NUCLEOTIDE ANALOGUE INCORPORATION.

### Kits and Articles of Manufacture

Kits are also a feature of the invention. The present invention provides kits that incorporate the compositions of the invention, optionally with additional useful reagents such as, including one or more enzyme, e.g., a DNA polymerase, an RNA polymerase, a uvrD, a Rep, a RecQ, a dnaB, a T4 gp41, a T7 gp4, or a reverse transcriptase, that can be unpackaged in a fashion to enable their use. The kits of the invention optionally include additional reagents, such as a control template nucleic acids, buffer solutions and/or salt solutions, including, e.g., divalent metal ions, i.e., Mg⁺⁺, Mn⁺⁺ and/or Fe⁺⁺, e.g., to hybridize molecular beacons to a concatamer of code units, to prepare hybridized molecular beacons for removal from a concatamer with a helicase, etc. Such kits also typically include a container to hold the kit components, instructions for use of the compositions, and other reagents in accordance with the methods, e.g., of removing molecular beacons from a DNA concatamer of code units.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A composition, comprising:
a) a nucleic acid;
b) at least one labeled hybridization probe which comprises a sequence complementary to a first subsequence of the nucleic acid; and,
c) an enzyme that exhibits a helicase activity, which enzyme is capable of dissociating the probe from the nucleic acid, which dissociation produces a signal.

2. The composition of claim 1, wherein the nucleic acid is at least partially single-stranded.

3. The composition of claim 1, wherein the nucleic acid is an RNA or a DNA.

4. (canceled)

5. The composition of claim 4, wherein the DNA comprises a concatenation of first code units and second code units, wherein first code units comprise first unique oligonucleotide sequences and second code units comprise second unique oligonucleotide sequences, such that each adenosine, cytosine, thymine, and guanine in a sequence of a target nucleic acid is represented by a code unit pair, such that a code unit sequence of the DNA represents the nucleotide sequence of the target nucleic acid.

6. The composition of claim 1, wherein the labeled hybridization probe is a first molecular beacon which comprises a first fluorophore at a first end, which fluorophore emits light at a first wavelength.

7. The composition of claim 1, wherein the first molecular beacon is hybridized to the first complementary subsequence of the nucleic acid.

8. The composition of claim 6, wherein the composition comprises at least one second molecular beacon that comprises a sequence complementary to a second subsequence of the nucleic acid and a second fluorophore at a first end that emits light at a second wavelength, wherein the second wavelength is different from the first wavelength of the first fluorophore of the first molecular beacon.

9. The composition of claim 8, wherein the second molecular beacon is hybridized to the second subsequence of the nucleic acid.

10. The composition of claim 8, wherein the first and second molecular beacons are hybridized to the nucleic acid in a head-to-tail arrangement.

11. The composition of claim 1, wherein the signal is a fluorescent signal.

12. The composition of claim 1, wherein the enzyme is a DNA polymerase, an RNA polymerase, a DNA helicase, an RNA helicase, a DNA/RNA helicase, a reverse transcriptase, or a ribosome.

13. The composition of claim 12, wherein the DNA helicase is a uvrD, a Rep, a RecQ, a dnaB, a T4 gp41, or a T7 gp4.

14. The composition of claim 12, wherein the DNA polymerase is a Taq polymerase.

15. The composition of claim 1, wherein the signal can be converted into nucleotide sequence information.

16. The composition of claim 1, wherein the composition is present on a planar surface, in a well, in a single-molecule reaction region, or in an observation volume.

17. The composition of claim 16, wherein the enzyme is immobilized on the planar surface, in the well, in a single-molecule reaction region, or in an observation volume.

18. The composition of claim 1, wherein the composition comprises ATP, GTP, CTP, TTP or UTP.

19. A method of determining the sequence of a template nucleic acid, the method comprising:
a) hybridizing one or more labeled hybridization probes to the template;
b) dissociating the probes from the template with an enzyme that exhibits probe-displacing activity to produce a signal;
c) detecting the signal or a sequence of signals; and,
d) converting the signal or sequence of signals into nucleotide sequence information, thus determining the sequence of the template nucleic acid.

20-28. (canceled)

29. A method of determining the sequence of a template nucleic acid, the method comprising:
a) providing a reaction mix comprising a thermostable enzyme that exhibits probe-displacing activity and one or more labeled hybridization probes annealed to the template;
b) dissociating the probes from the template with the enzyme to produce a signal;
c) detecting the signal or a sequence of signals;
d) converting the signal or sequence of signals into nucleotide sequence information;
e) increasing the temperature of the reaction mix to dissociate the remaining probes from the template and to release the enzyme from the template; and
f) lowering the temperature of the reaction mix to allow rehybridization of the probes to the template.

30. (canceled)

31. A sequencing system, comprising:
a reaction region which contains a template nucleic acid to which a set of molecular beacons has been hybridized and an enzyme, wherein the enzyme comprises a probe displacing activity and is capable of sequential removal of the molecular beacons from the template nucleic acid;
a detector configured to detect a sequence of fluorescent signals produced by the sequential removal of the molecular beacons by the enzyme in the reaction region; and,
a conversion module that is capable of converting the sequence of fluorescent signals into nucleotide sequence information.
